(19) United States
(12) Patent Application Publication
(10) Pub. No.: US 2013/0118996 A1
(43) Pub. Date: May 16, 2013

(54) HEXAHYDROTRIAZINES, SYNTHESIS AND USE

(71) Applicant: Gregory Kaplan, Richboro, PA (US)

(72) Inventor: Gregory Kaplan, Richboro, PA (US)

(73) Assignee: GENERAL ELECTRIC COMPANY, Schenectady, NY (US)

(21) Appl. No.: 13/293,691

(22) Filed: Nov. 10, 2011

(51) Int. Cl. C10G 17/04; C07D 251/04

(52) U.S. Cl. 210/749; 544/215

(57) ABSTRACT

Methods for making asymmetrical triazines are provided. The methods comprise first forming a mixture of at least two primary amines then reacting the mixture with an aldehyde. Methods for removing sulfides from hydrocarbon streams are also provided. The triazines may be added to the hydrocarbon stream in a molar ratio of triazine:H₂S of about 10:1 to about 1:2.

### FIELD OF INVENTION

The invention pertains to methods and chemical compositions for reacting with hydrogen sulfide (H₂S), and more particularly, for scavenging H₂S from hydrocarbon streams in the petroleum and natural gas industries.

### BACKGROUND OF THE INVENTION

Hydrogen sulfide, or H₂S, is a clear, toxic gas with a foul odor. It is also highly flammable. The Environmental Protection Agency and other regulatory agencies worldwide strictly control the release of H₂S into the environment. H₂S is often present in crude oil and natural gas reserves and must be removed before making commercial use of such reserves. The H₂S concentration in these reserves prior to treatment typically varies with location and is usually higher in natural gas than in crude oil reserves. In natural gas reserves, for example, H₂S may vary from less than 100 ppm to 3,000 ppm. Permitted H₂S levels will also vary by location. The U.S. limits H₂S in natural gas pipelines to 4 ppm per 100 standard cubic feet (0.3 gr/100 scf).

Generally, hydrocarbon streams are treated to remove H₂S, mercaptans, or organic sulfides by using chemicals that will react with sulfide contaminants. These chemicals are called scavengers, or sweetening agents. Hexahydrotriazines, commonly called “triazines,” are frequently used as H₂S, mercaptan, and organic sulfide scavengers. Triazines are a water-soluble species characterized as having a benzene ring structure in which three nitrogens with alkyl radicals replace three carbon-hydrogen units. Triazines scavenge H₂S through the following reaction:

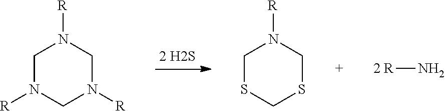

wherein R is a straight or branched alkyl radical, including substituted alkyl radicals, with the general formula CₙH₂ₙ₊₁.

Most hydrocarbon reserves are treated continuously near the wellhead, though treating hydrocarbons in a batch or similar application elsewhere is not uncommon. Continuous treatment installations near the wellhead inject scavengers, like triazines, directly into the hydrocarbon pipeline. The injection system typically includes a chemical injection pump and piping tees or atomization nozzles to introduce the triazines into the pipeline. Based on the stoichiometry of the scavenging reaction, a molar ratio of triazine to H₂S of 1:2 is ideal. The amount of triazine actually required, however, will vary depending on a variety of factors including the amount of H₂S in the well, permissible H₂S limits, the well flow rate, temperature, etc. and may be determined by those of ordinary skill in the art. Thus, to effectively treat the hydrocarbon stream, the triazine:H₂S molar ratio may vary from about 10:1 to about 1:2. A length of the pipeline is provided to allow for contact between the scavenger and the H₂S. At the end of the length, the spent scavenger is separated from the hydrocarbon stream and the hydrocarbon stream moves on for further processing or use.

The most common method of making triazines, including those used in H₂S scavenging applications is to add a primary amine to an aldehyde. The aldehyde may be either a water solution of formaldehyde or solid paraformaldehyde. Primary amines are compounds where one of three hydrogen atoms in ammonia is replaced by an alkyl. Examples of primary amines include methylamine, ethanolamine, monoethanolamine and methoxypropylamine The most commonly used triazines are made from monoethanolamine (MEA). The resulting triazine is hexahydro-1,3,5-tris(hydroxyethyl)-s-triazine, or triazinetriethanol, and has the structure:

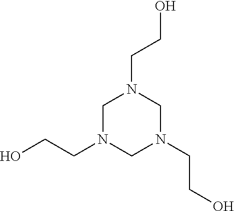 [SMILES: OCCN1CN(CCO)CN(CCO)C1]

The triazine structure can be altered through the use of different types of primary amines. Thus using methoxypropylamine (MOPA) results in hexahydro-1,3,5-tris(methoxypropyl)-s-triazine, and has the structure:

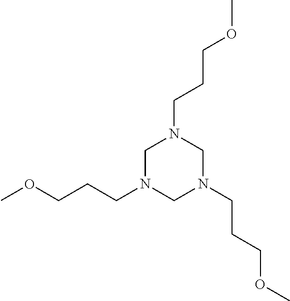

Hexahydro-1,3,5-tris(hydroxyethyl)-s-triazine triazine has a high freezing point around −40° F. (−40° C.). This freezing point is not low enough for many hydrocarbon reserves in the northern hemisphere where winter temperatures may fall below −40° F. (−40° C.). Once temperatures reach below −40° F. (−40° C.) in scavenging applications utilizing these triazines, they cannot be injected into the hydrocarbon stream using typical methods. This is true for the northern United States, Canada, Russia, Kazakhstan, northern China, and Norway. In these areas, the symmetrical triazines are diluted with expensive and flammable solvents and antifreezes, such as methanol or ethylene glycol, to prevent freezing and achieve low temperature handling capability. This not only minimizes H₂S scavenging activity, but also increases the cost to purchase and ship the symmetrical triazines used in these areas.

Hexahydro-1,3,5-tris(methoxypropyl)-s-triazine has a freezing point below −40° F. (−40° C.); however, it is less effective as an H₂S scavenger.

### SUMMARY OF THE INVENTION

In one embodiment of the invention, a method of making at least one asymmetrical hexahydrotriazine or “triazine” is provided. The asymmetrical triazine is made by first forming a mixture of at least two primary amines. Then the mixture of primary amines is reacted with an aldehyde compound. The aldehyde compound may be an aqueous formaldehyde solution or solid paraformaldehyde. In another embodiment of the invention, the primary amines include monoethanolamine (MEA) and methoxypropylamine (MOPA) in a molar ratio of about 2:1.

In another embodiment of the invention, a method is provided for removing sulfides, including H₂S, mercaptans, and organic sulfides, from liquid or gaseous hydrocarbon streams. The method comprises contacting sulfides in the hydrocarbon stream with one or more asymmetric triazines. The method may be used in low temperature applications without adding solvents and antifreezes.

In yet another aspect of the invention, an asymmetrical triazine is provided. These asymmetrical triazines have different alkyl groups attached to the nitrogen atoms. The alkyl groups may be straight or branched. In yet another aspect of the invention, the asymmetrical triazine has both MEA and MOPA primary amines incorporated into its structure.

The present invention and its advantages over the prior art will become apparent upon reading the following detailed description and the appended claims.

### DETAILED DESCRIPTION OF EXEMPLARY EMBODIMENTS

Exemplary embodiments include asymmetrical triazines and methods for manufacturing such triazines. Other embodiments utilize asymmetrical triazines to scavenge H₂S at low temperatures without adding expensive and flammable solvents and antifreezes, such as methanol or ethylene glycol. The embodiments are described in conjunction with the following examples.

The first exemplary embodiment discloses a method for making asymmetrical triazine by first forming a mixture of two or more different primary amines Examples of primary amines include methylamine, ethanolamine, monoethanolamine and methoxypropylamine The primary amine mixture may include MEA and MOPA. The molar ratio of the two primary amines may vary. In one embodiment, the molar ratio is about 2:1. The mixture of primary amines is then reacted with an aldehyde compound. Aldehyde compounds include formaldehyde in a water solution and solid paraformaldehyde. The molar ratio of the amine mixture to the aldehyde compound is about 1:1. The resulting triazines have the general structure and formula I:

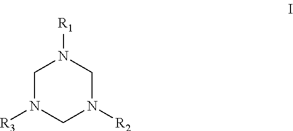

wherein R₁, R₂, and R₃ are chosen from alkyls, hydroxyl-substituted alkyls, and alkoxy-substituted alkyl of 1 to 10 carbon atoms, with the proviso that R₂, R₂, and R₃ are not all the same. The alkyl groups may be straight or branched alkyl groups, including, but not limited to, methyl, ethyl, propyl, butyl, ethyl hydroxyl, and methoxypropyl.

Another exemplary embodiment discloses using asymmetrical triazines to remove sulfides, including H₂S and mercaptans, from hydrocarbon streams. The hydrocarbon streams may be gaseous or liquid streams. The sulfides are contacted with at least one asymmetrical triazine, including triazines that incorporate both the MEA and MOPA structures. The asymmetrical triazines may be added in an asymmetrical triazine:H₂S molar ratio of about 10:1. In another embodiment, the molar ratio of asymmetrical triazine:H₂S is about 5:1 and in another embodiment, the molar ratio is about 1:2.

In another embodiment, asymmetrical triazines are used to remove sulfides, including H₂S and mercaptans, from hydrocarbon streams at low temperatures without adding solvents or antifreezes. The hydrocarbon streams may be gaseous or liquid streams. The sulfides are contacted with at least one asymmetrical triazine, including triazines that incorporate both the MEA and MOPA structures. The amount of triazine added will depend on the application and amount of sulfide scavenging required and may vary from about 10:1 to about 1:2. The asymmetrical triazines are added in an asymmetrical triazine:H₂S molar ratio of about 10:1. In one embodiment, the molar ratio of asymmetrical triazine:H₂S is about 5:1 and in another embodiment, the molar ratio is about 1:2. In another embodiment, the ratio is about 2.5:1 to about 3.5:1. In another embodiment, the resulting triazines incorporate both the MEA and MOPA structures, producing asymmetrical triazines with at least one of the following formula and structures II or III:

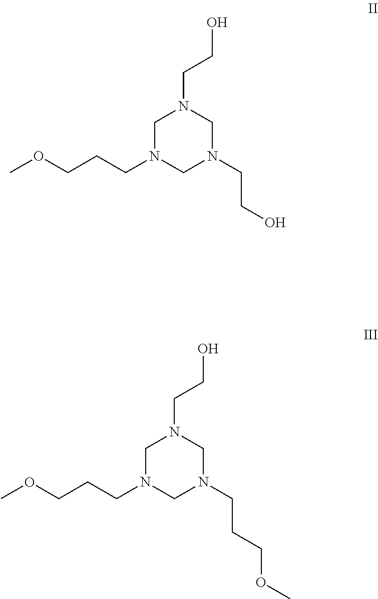

These asymmetrical triazines are beneficial because they have a freezing point around −60° F. (−51° C.). The commonly used triazines have a high freezing point around −40° F. (−40° C.) and require dilution with expensive and flammable solvents and antifreezes, such as methanol or ethylene glycol, to prevent freezing and achieve low temperature handling capability. Thus, these asymmetrical triazines can be used for H₂S scavenging at low temperatures without adding expensive and flammable solvents and antifreezes, such as methanol or ethylene glycol.

### EXAMPLES

The method of manufacture of an asymmetrical triazine was compared to the method of manufacture of a symmetrical triazine. The effects of these two types of triazines on a hydrocarbon stream containing H₂S were also compared. The following examples illustrate these comparisons.

### Example 1

Example 1 utilizes a mixture of two or more primary amines, monoethanolamine (MEA) and methoxypropylamine (MOPA). The molar ratio of MEA to MOPA is 2:1 but the molar ratio may vary. In Example 1, the asymmetrical triazine was made in a flask equipped with a stirrer, condenser, and temperature control device. The flask was charged with 1 Mole (31.25 gm) of 96% pure paraformaldehyde. The primary amines were premixed in a separate container. The primary amine mixture included 0.66 Mole (40.26 gm) monoethanolamine (MEA) and 0.34 (30.0 gm) of methoxypropylamine (MOPA). The primary amine mixture was then added drop-wise to the flask containing the paraformaldehyde while controlling the temperature in the flask to below 50° C. After the mixture was added, the contents of the flask were stirred for 1 hour while the temperature of the flask was maintained at 80° C. After one hour, 102 grams of asymmetrical triazine was collected. The product was a transparent single phase solution. The asymmetrical triazine that was produced had a freezing point of −60° F. (−51° C.).

### Example 2

The efficacy of the product produced in Example 1 was tested in Example 2. In this example, 200 ml of a light hydrocarbon mixture having 2000 ppm of H₂S level in the head space was placed in a 1-liter bottle. Next, 5500 ppm of the asymmetrical triazine produced in Example 1 was added to the 1-liter bottle. After stirring for 30 minutes at room temperature, the H₂S level in the head space was reduced to 100 ppm.

### Example 3

In this example, 200 ml of a light hydrocarbon mixture having 2000 ppm of H₂S level in the head space was placed in a 1-liter bottle. Next, 6500 ppm of the asymmetrical triazine produced in Example 1 was added to the 1-liter bottle. After stirring for 30 minutes at room temperature, the H₂S level in the head space was reduced to 6 ppm.

### Example 4

For comparison to Example 1, in Example 4, a symmetrical triazine was made in a flask equipped with a stirrer, condenser, and temperature control device. The flask was charged with 1 Mole (31.25 gm) of 96% pure paraformaldehyde. 1 Mole (61 gm) of monoethanolamine (MEA) was added drop-wise to the flask containing the paraformaldehyde while controlling the temperature in the flask to below 50° C. After the all the MEA was added, the contents of the flask were stirred for 1 hour while the temperature of the flask was maintained at 80° C. After one hour, 92 grams of symmetrical triazine was collected. The symmetrical triazine that was produced with the above method had a freezing point of −40° F. (−40° C.).

### Example 5

For comparison to our Examples 2 and 3, the efficacy of the product produced in Example 4 was tested in Example 5. In this example, 200 ml of a light hydrocarbon mixture having 2000 ppm of H₂S level in the head space was placed in a 1-liter bottle. Next, 5500 ppm of the symmetrical triazine produced in Example 4 was added to the 1-liter bottle. After stirring for 30 minutes at room temperature, the H₂S level in the head space was reduced to 2 ppm.

### Example 6

For comparison to the product produced in Example 1, a mixture of hexahydro-1,3,5-tris(hydroxyethyl)-s-triazine (MEA triazine) and hexahydro-1,3,5-tris(methoxypropyl)-s-triazine (MOPA triazine) was made. First a 75% MEA triazine solution in water was made. Added to that solution was a 75% MOPA triazine solution in water. The solutions were stirred. Unlike Example 1, which was a transparent single phase solution, the solutions in Example 6 separated into two immiscible layers. This clearly supports claims of an asymmetrical triazine with both MEA and MOPA structures.

This written description uses examples to disclose the invention, including the best mode, and also to enable any person skilled in the art to practice the invention, including making and using any devices or systems and performing any incorporated processes. These examples are merely illustrative and do not limit the invention in any manner For example, although the asymmetrical triazine synthesis and scavenging conditions in the illustrative examples list specific temperatures, these reactions can occur at almost any temperature. The patentable scope of the invention is defined by the claims, and may include other examples that occur to those skilled in the art. These other examples are intended to be within the scope of the claims if they have structural elements that do not differ from the literal language of the claims, or if they include equivalent structural elements with insubstantial differences from the literal language of the claims.

What is claimed is:

1. A method for making at least one asymmetrical hexahydrotriazine comprising:
(a) forming a first mixture comprising at least two primary amines; and
(b) reacting said first mixture with an aldehyde compound.

2. The method of claim 1, wherein at least one of said two primary amines includes monoethanolamine or methoxypropylamine

3. The method of claim 2, wherein a molar ratio of monoethanolamine to methoxypropylamine ranges from 20:1 to 1:20.

4. The method of claim 1, wherein a molar ratio of said first mixture to said aldehyde compound is about 1:1.

5. The method of claim 1, wherein said aldehyde compound is formaldehyde in a water solution.

6. The method of claim 1, wherein said aldehyde compound is solid paraformaldehyde.

7. A method for removing sulfides including hydrogen sulfide, mercaptans, or organic sulfides from hydrocarbon streams comprising:
(a) providing a hydrocarbon stream, wherein said hydrocarbon stream is a gas, liquid, or combination thereof; and
(b) contacting said sulfides in said hydrocarbon stream with at least one asymmetrical hexahydrotriazine.

8. The method of claim 7, wherein said asymmetrical hexahydrotriazine is added to said hydrocarbon stream in a molar ratio of hexahydrotriazine:H₂S of about 10:1.

9. The method of claim 7, wherein said asymmetrical hexahydrotriazine is added to said hydrocarbon stream in a molar ratio of hexahydrotriazine:H₂S of about 5:1.

10. The method of claim 7, wherein said asymmetrical hexahydrotriazine is added to said hydrocarbon stream in a molar ratio of hexahydrotriazine:H₂S of about 1:2.

11. The method of claim 7, wherein said sulfides are removed from said hydrocarbon stream without adding any solvents or antifreezes.

12. The method of claim 7, wherein said sulfides are removed from said hydrocarbon steam at temperatures of about −40° C. and below.

13. The method of claim 7, wherein said hexahydrotriazine has a structure and formula: 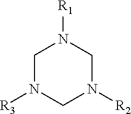
wherein R₁, R₂, and R₃ are chosen from alkyls, hydroxyl-substituted alkyls, and alkoxy-substituted alkyl of 1 to 10 carbon atoms, with the proviso that R₁, R₂, and R₃ are not all the same.

14. The method of claim 13, wherein the alkyl groups are straight or branched alkyl groups.

15. The method of claim 13, wherein R₁ is ethyl hydroxyl and R₂ and R₃ are methoxypropyl.

16. The method of claim 13, wherein R₁ and R₂ are ethyl hydroxyl and R₃ is methoxypropyl.

17. The method of claim 13, wherein said hexahydrotriazine has an asymmetrical structure and formula:

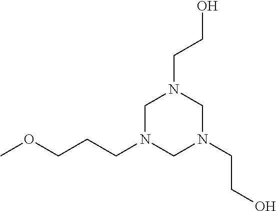 [SMILES: COCCCN1CN(CCO)CN(CCO)C1]

18. The method of claim 13, wherein said hexahydrotriazine has an asymmetrical structure and formula:

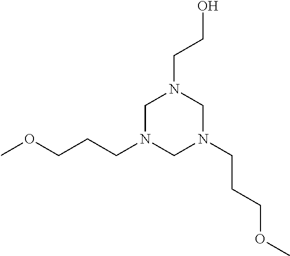

19. A hexahydrotriazine having an asymmetrical structure and formula: 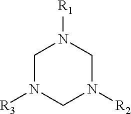
wherein R₁, R₂, and R₃ are chosen from alkyls, hydroxyl-substituted alkyls, and alkoxy-substituted alkyl of 1 to 10 carbon atoms, with the proviso that R₁, R₂, and R₃ are not all the same.

20. The hexahydrotriazine of claim 19, wherein said alkyl radicals are straight or branched alkyl groups.

21. The hexahydrotriazine of claim 19, wherein R₁ is ethyl hydroxyl and R₂ and R₃ are methoxypropyl.

22. The hexahydrotriazine of claim 19, wherein R₁ and R₂ are ethyl hydroxyl and R₃ is methoxypropyl.

23. A hexahydrotriazine having an asymmetrical structure and formula:

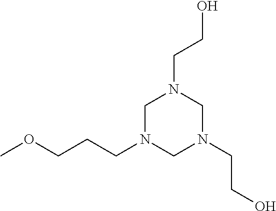 [SMILES: COCCCN1CN(CCO)CN(CCO)C1]

24. A hexahydrotriazine having an asymmetrical structure and formula: [chemical structure]
